(19) United States
(12) Patent Application Publication
(10) Pub. No.: US 2002/0019991 A1
(43) Pub. Date: Feb. 14, 2002

(54) COMPOSITIONS CONTAINING AN ALPHA 1,2-FUCOSE LINKAGE AND USES THEREOF

(75) Inventors: PEDRO A. PRIETO, WEST WORTHINGTON, OH (US); GUILLERMO M. RUIZ-PALACIOS, DELEGACION TLALPAN (MX)

Correspondence Address: ABBOTT LABORATORIES, DEPT. 377 - AP6D-2, 100 ABBOTT PARK ROAD, ABBOTT PARK, IL, 60064-6050, US

(73) Assignee: ABBOTT LABORATORIES

(21) Appl. No.: 09/070,177

(22) Filed: Apr. 30, 1998

(51) Int. Cl. G01N 33/00

(52) U.S. Cl. 800/3

(57) ABSTRACT

The subject intention relates to compositions containing at least one fucose residue in an α1-2 linkage and uses thereof. In particular, such compositions can be used in the treatment and prevention of gastrointestinal infections caused by, for example, Escherichia coil and Vibrio cholerae. The subject invention also encompasses methods of screening for the above compositions. Additionally, the subject invention includes vaccines.

### BACKGROUND OF THE INVENTION

1. Technical Field

The subject invention relates to compositions comprising at least one fucose residue in an α1-2 linkage and uses thereof. In particular, such compositions can be used in the treatment and prevention of gastrointestinal infections caused by, for example, Escherichia coli and Vibrio cholerae. The subject invention also encompasses methods of screening for the above compositions. Additionally, the subject invention includes vaccines.

2. Background Information

Diarrheal diseases are a major cause of morbidity and mortality worldwide, both in children and adults, accounting for an estimated 5 to 10 million deaths each year. Disease burden is high especially among children living in developing countries (Calva et al., Lancet 1:503-506 (1988); Black et al., Am. J. Epidemiol. 129:785-799 (1989)). Two pathogens are of special importance, namely Vibrio cholerae and enterohemorrhagic Escherichia coli (Sanchez, J. L., Lancet 349:1825-1830 (1997); Glass et al., Science 256:1524-1525 (1992); Sharp et al., PHLS Microbiology Digest 12:134-140 (1995)).

V. cholerae is a major cause of epidemic diarrhea in developing regions. Pandemias have expanded to the New World, during this decade, and infection by V. cholerae is considered one of the re-emerging infectious diseases. Of particular interest is the fact that a higher susceptibility to V. cholerae infection has been observed in individuals with the O (H) blood group antigen (Glass et al., Am. J. Epidemiol. 121(6):791-796 (1985)).

There is now considerable effort dedicated to the prevention and control of V. cholerae infection, especially since a new serotype (i.e., 0139) has emerged (Sack et al., Curr. Clin. Tropics Infect. Dis. 16:172-193 (1996); Morris et al., Infect. Agents Dis. 4:41-46 (1995); Scas et al., Int. J. Infect. Dis. 1:37-46 (1996)). The use of antibiotics in this infection is of limited value, and the emergence of resistance is a major drawback, with oral rehydration therapy still being the cornerstone of treatment. The development of compounds that prevent attachment of V. cholerae to the intestinal wall, such as oligosaccharide receptor analogs, and the development of vaccines, using adhesins as antigens, may therefore be useful in the treatment and prevention of this major invention.

Enterohemorrhagic E. coli (EHEC) is now considered as an emerging pathogen of worldwide importance, particularly in industrialized countries (Sharp et al., PHLS Microbiology Digest 12:134-140 (1995); Centers for Disease Control and Prevention. Escherichia coli 0157:h7 outbreak linked to commercially distributed dry-cured salami, Washington and California. Morbid Mortal Weekly Rep. 44:157-160 (1994)). EHEC infection has a very high mortality rate, and the clinical spectrum in humans is wide, including non-bloody diarrhea, hemorrhagic colitis, and hemolytic uremic syndrome (HUS). EHEC has been isolated in multiple outbreaks associated with the consumption of contaminated meat. Serotype 0157:H7 is the serotype most frequently associated with HUS, although other serotypes have also been associated with this condition (Griffin et al., Epidemiol. Rev. 13:60-98 (1991)). EHEC produces large amounts of a shig-like cytotoxin which is considered to be the major pathogenic factor related to HUS (Thomas et al., Epidemiol. Infect. 110:591-600 (1993); Willshaw et al., Emerg. Infect. Dis. 4:561-565 (1997)). Attachment of EHEC to epithelium of the terminal ileum, cecum and colon is a complex process that occurs in multiple stages and may be similar to the process used by enteropathogenic E. coli (EPEC).

Several bacterial components have been implicated in the adherence of EHEC to epithelial cells, producing characteristic attaching and effacing (A/E) lesions in these cells. However, cell receptors for EHEC are not yet well characterized. The identification of attachment-related epithelial receptors may aide in the design of strategies for the control of the infection. It is possible that, as in other enteropathogens, oligosaccharides may be important as receptors for this bacteria, and the identification of oligosaccharide receptor analogs may be a potentially valuable tool for the prevention and treatment of this very serious infection.

In view of the above, methods of preventing and treating V. cholerae and enterohemorrhagic E. coli infections as well as compounds for achieving this purpose are needed in order to save lives. The present invention provides such methods and compounds.

### SUMMARY OF THE INVENTION

The present invention relates to a pharmaceutical composition comprising at least one fucose residue in an α1-2 linkage and a pharmaceutically acceptable carrier. The at least one fucose residue in an α1-2 linkage may be present in a compound selected from the group consisting of, for example, 2′-fucosyllactose, difucosyllactose, Fucα1-2Galβ1-4[Fucα1-3]Glc, glycoproteins or glycopeptides containing the structure Fucα1-2Galβ1-4Glc Nac β1-3 GM1-Fuc (Fucα1-2Galβ1-3GalNac), glycolipids and fucosylated derivatives of neutral glycolipids.

Additionally, the present invention encompasses a nutritional composition comprising at least one fucose residue in an α1-2 linkage, at least one protein not found in human breast milk, and at least one member selected from the group consisting of an edible fat, a carbohydrate, a protein, a vitamin and a mineral. Again, the at least one fucose residue in the α1-2 linkage may be present in a compound selected from the group consisting of, for example, 2′-fucosyllactose, difucosyllactose, Fucα1-2Galβ1-4[Fucα1-3]Glc, glycoproteins or glycopeptides containing the structure Fucα1-2Galβ1-4Glc Nac β1-3 GM1-Fuc (Fucα1-2Galβ1-3GalNac), glycolipids and fucosylated derivatives of neutral glycolipids. This nutritional composition may be, for example, an infant formula.

Furthermore, the present invention also includes a rehydration solution comprising the compositions described above.

Moreover, the present invention also encompasses a method of preventing or treating diarrhea or enterocolitis in a patient. This method comprises administering a composition comprising at least one fucose residue in an α1-2 linkage to a patient in need of such prevention or treatment. The composition is administered in an amount sufficient to effect the treatment or prevention. The composition may be 15 administered to a human or to an animal. The diarrhea or enterocolitis, for example, NEC, may be caused by a microorganism selected from the group consisting of Escherichia coli and Vibrio cholerae.

The present invention also includes a method of screening for a composition which prevents the attachment of E. coli or V. cholerae to a host cell receptor. This method comprises the steps of: a) exposing the composition in question to the host cell receptor; b) adding Escherichia coli or Vibrio cholerae to the composition of step (a) and the host cell receptor; and c) determining whether inhibition of binding of the cells to the host cell receptor occurs, inhibition indicating the presence of a composition which binds to the host cell receptor and prevents attachment of said E. coli or V. cholerae to the host cell receptor. The receptor may comprise a fucosylated blood group antigen. The fucosylated blood group antigen may be, for example, H-2.

Additionally, the present invention also includes a vaccine which comprises at least one protein which binds to at least one fucose residue in an α1-2 linkage and a physiologically acceptable adjuvant. The vaccine may be administered, for example, subcutaneously or intramuscularly.

Also, the present invention includes a method of screening for a composition which prevents the attachment of E. coli or V. cholerae to a mammalian cell receptor. This method comprises the steps of: a) constructing a transgenic mammalian embryo which, upon birth, produces a composition comprising at least one fucose residue in an α1-2 linkage; b) implanting the transgenic mammalian embryo into a recipient adult female; c) allowing gestation and birth to occur; d) challenging the resulting mammal with E. coli or V. cholerae; and e) determining whether infection develops in the resulting mammal, lack of infection indicating that the composition expressed by the resulting mammal prevents attachment of E. coli or V. cholerae to the receptor of cells of the resulting mammal.

The present invention also includes another method of screening for a composition which prevents the attachment of E. coli or V. cholerae to a host cell receptor. This method comprises the steps of: a) exposing transfected, mammalian cells expressing a neoglyconjugate to E. coli or V. cholerae; b) determining whether binding has occurred between the mammalian cells and the E. coli or V. cholerae, a high degree of binding inhibition relative to a control indicating that the neoglyconjugate prevents attachment of the E. coli or V. cholerae to the receptor of the mammalian cells.

Furthermore, the present invention includes an additional method of screening for a composition which prevents the attachment of E. coli or V. cholerae to a host cell receptor. This method comprises the steps of: a) purifying a glycoconjugate comprising at least one fucose residue in an α1-2 linkage from a mammalian cell; b) immobilizing the glycoconjugate on a solid support; c) exposing the immobilized glycoconjugate to E. coli cells or V. cholerae cells; d) adding a composition of interest to the immobilized glycoconjugate and E. coli cells or V. cholerae cells; d) determining whether binding occurs between the immobilized glycoconjugate and the E. coli cells or V. cholerae cells, lack of binding indicating a composition which prevents the attachment of E. coli cells or V. cholerae cells to a host cell receptor.

Also, the present invention includes a further method of screening for a composition which prevents the attachment of E. coli or V. cholerae to a receptor of mammalian cells. This method comprises the steps of: a) constructing a transgenic, mammalian embryo which, after birth, produces a composition comprising at least one fucose residue in an α1-2 linkage; b) implanting the transgenic, mammalian embryo into a recipient female; c) allowing gestation and birth to occur; d) allowing the resulting transgenic mammal to mate and produce offspring; e) allowing the offspring to suckle on milk produced by the transgenic mammals; f) challenging the offspring with E. coli cells or V. cholerae cells; and g) determining whether infection occurs, lack of infection indicating a composition present in the milk of the transgenic mammal which prevents the attachment of E. coli or V. cholerae to a receptor of cells of the offspring.

The present invention also includes a method of screening pathogenic microorganisms from non-pathogenic microorganisms. This method comprises the steps of: a) isolating a microorganism of interest; b) exposing the microorganism to a glyconjugate receptor comprising at least one fucose residue in an α1-2 linkage, wherein the receptor binds only to pathogenic microorganisms; and c) determining whether binding occurs between the glycoconjugate receptor and the microorganism of interest, binding indicating that the microrganism is pathogenic and non-binding indicating that the microorganism is non-pathogenic. All U.S. patents and publications referred to herein are hereby incorporated in their entirety by reference.

### BRIEF DESCRIPTION OF THE DRAWINGS

FIG. 1 illustrates Western blots of commercially available glycoconjugates probed with labeled Campylobacter jejuni. Panel A represents an invasive strain, and Panel B represents a non-invasive strain.

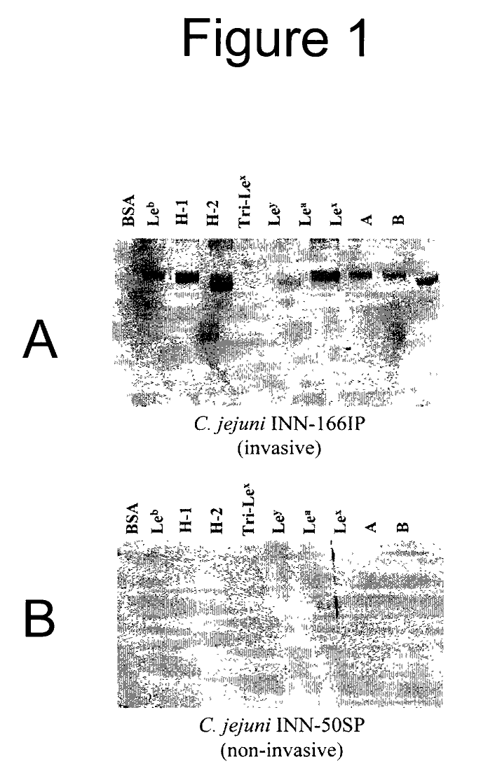

FIG. 2 represents Western blots of commercially available glycoconjugates probed with labeled Vibrio cholerae (Panel A), labeled Escherichia coli (Panel B) and Enterohemorrhagic Escherichia coli (Panel C).

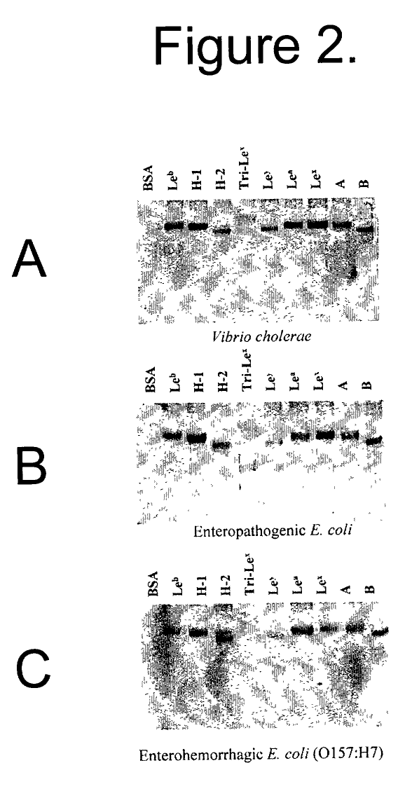

FIG. 3 illustrates specific binding inhibition of V. cholerae to H-2-neoglyconjugates by monoclonal antibody against H-2 antibody.

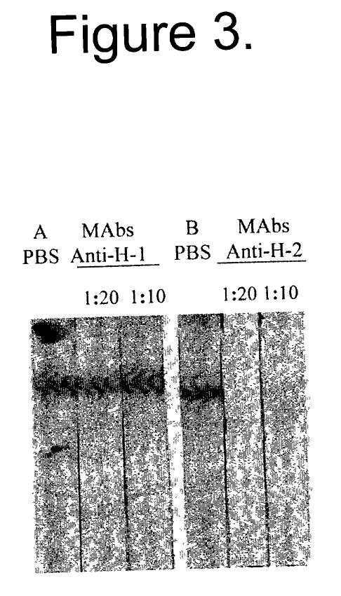

FIG. 4 illustrates V. cholerae binding to wild-type Chinese Hamster Ovary (CHO) cells (Panel A) and CHO cells transfected with the human αT1,2-fucosyltransferase gene (Panel B. Panel C represents an electron scanning microscope image of selected cells in Panel B.

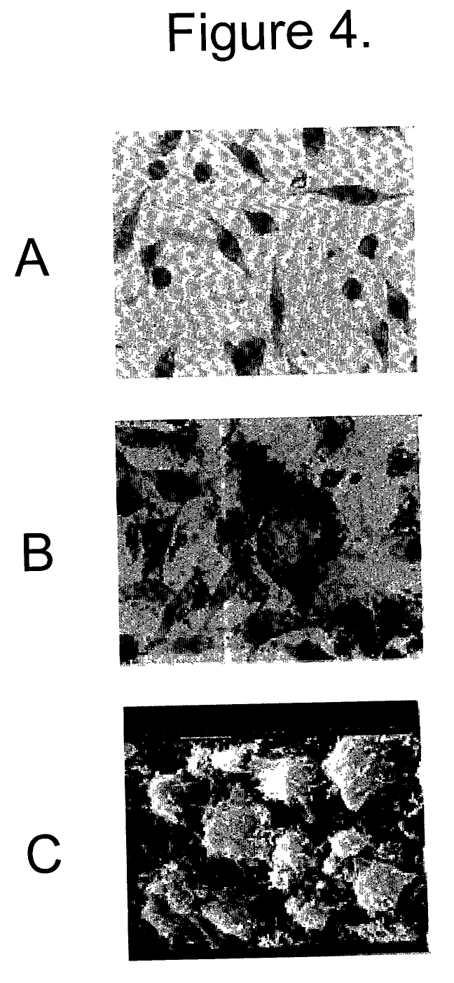

FIG. 5 represents the binding kinetics of two different dilutions of V. cholerae to CHO cells transfected with the human α-1,2-fucosyltransferase H.

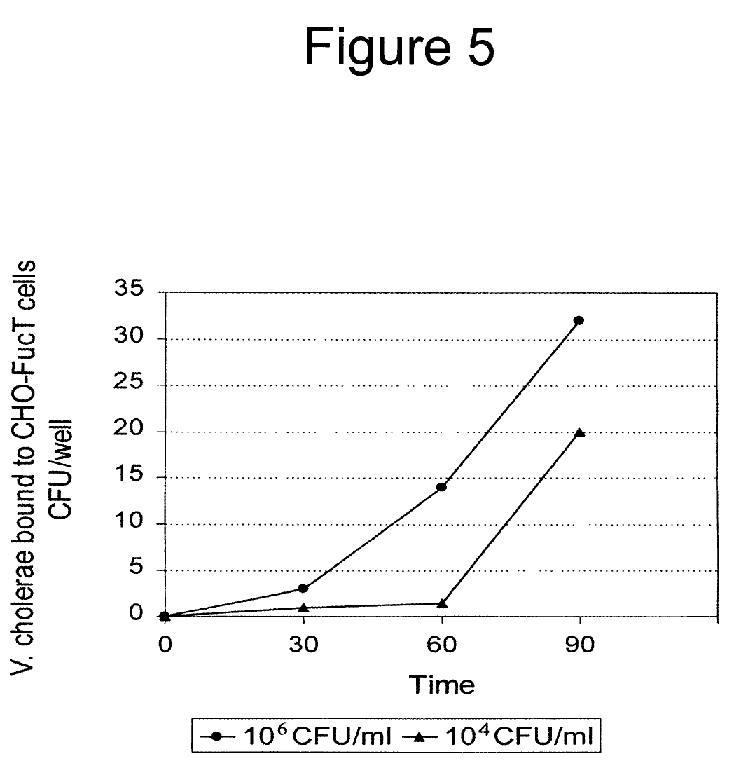

FIG. 6 illustrates the effect of 2′-fucosyllactose on the binding of pathogenic bacterial cells to CHO cells transfected with the human α-1,2-fucosyltransferase “H”. Panel A represents the binding of Vibrio cholerae, and Panel B represents the binding of enteropathogenic E. coli.

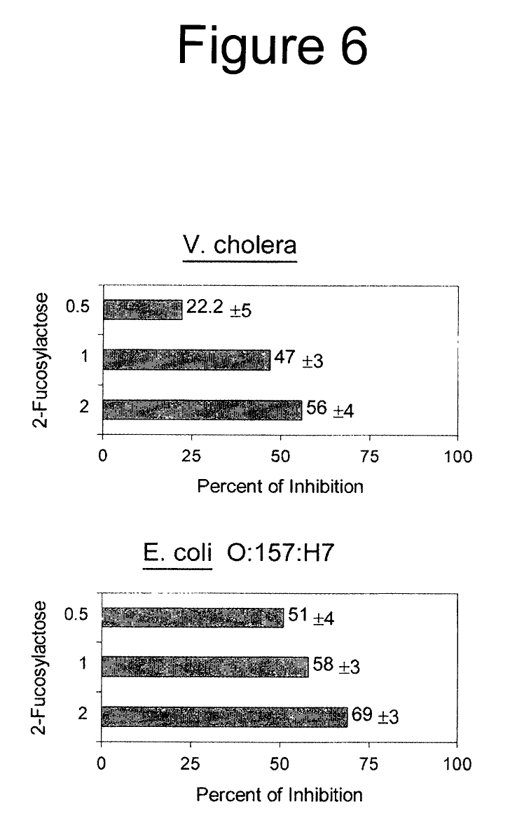

FIG. 7 represents a Western blot of electrophoresed V. cholerae proteins probed with dioxygenin-labeled neoglycoconjugate H-2 which contained Fuc α1-2 residues.

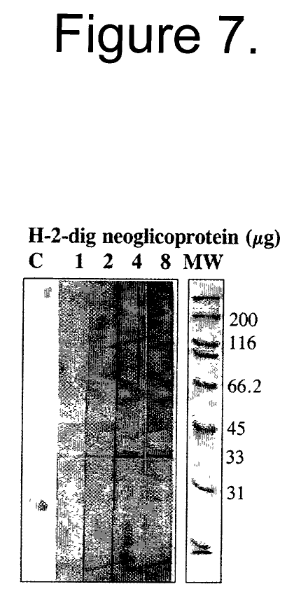

### DETAILED DESCRIPTION OF THE INVENTION

The subject invention relates to compositions comprising at least one fucose residue in an α1-2 linkage and to methods of use thereof. In particular, such compositions may be used in the treatment or prevention of various gastrointestinal infections or conditions caused by various microorganisms such as, for example, E. coli and V. cholerae. Additionally, the present invention includes methods of screening pharmaceuticals, comprising at least one fucose residue in an α1-2 linkage, for the ability to inhibit the binding of microorganisms such as for example, E. coli and V. cholerae, to host cells. Furthermore, the present invention also encompasses vaccines which comprise proteins obtained from gastrointestinal pathogens, for example, E. coli and V. cholerae, which bind fucose residues in α1-2 linkages.

More specifically, the compositions of the present invention comprise at least one fucose residue in an α1-2 linkage to, for example, galactose. Such a linkage typically occurs at the non-reducing ends of oligosaccharides and at terminal galactose residues of other glycoconjugates. Furthermore, the linkage may also be found in connection with monosaccharide residues other than galactose.

Compositions which include at least one fucose residue in an α1,2 linkage include, for example, 2′-fucosyllactose (Fucα1-2Galβ1-4Glc), difucosyllactose, Fucα1-2Galβ1-4[Fucα1-3]Glc, glycoproteins or glycopeptides containing the structure Fucα1-2Galβ1-4Glc Nac β1-3 (Prieto et al., J. Biol. Chem. 272(4):2089-2097 (1997)), glycolipids such as fucosylated derivatives of the gangliosides GM1, GM2 and GM3, fucosylated derivatives of neutral glycolipids such as lactosyl ceramide, other glycolipids of the ganglio and lacto series (Methods in Enzymology, Complex Carbohydrates, Part D, 1982, Vol. 83, pages 145-146), fucose rich oligosaccharides, from freshwater or marine algae or kelp, such as fucoidans, and other naturally occurring glycoconjugates which comprise fuc α1-2 linkages (Kurome et al., Phytochemistry 30(2):535-39 (1991)).

Further, the present invention also comprises compositions comprising analogues which mimic the fuc α1-2 epitope in such a way that their affinity for the carbohydrate binding domain of enteropathogenic bacteria is equal to or greater than the compositions described above comprising at least one fucose residue in an α1-2 linkage. Compositions which comprise the above chemical entities are also encompassed by the present invention (e.g., glycoproteins).

The composition containing the linkage of interest may be produced recombinantly, for example, by transgenic means, chemically synthesized or purified from native sources such as from algae, fungi, bacteria, human milk, mammalian intestine or other mammalian tissue.

Additionally, the compositions of the present invention may contain a pharmaceutically acceptable carrier in addition to the α1,2-fucose linkage or residue. As used herein, the term “pharmaceutically acceptable carrier” encompasses any of the standard pharmaceutical carriers, such as phosphate buffered saline solution, mixtures of ethanol in water, water and emulsions such as an oil/water or water/oil emulsion, as well as various wetting agents or excipients.

The compositions of the present invention may be administered by any method known to those of ordinary skill in the art including, but not limited to, parenteral or enteral administration. Examples of such administration include subcutaneous, intramuscular, topical, oral, intravenous, aerosal, tube (e.g., naso-gastric tube), and direct infusion into the GI tract or stomach. Administration will be in such a dosage as to effect the desired outcome. Such a dosage may be readily determined by one of ordinary skill in the art and depends upon such factors as, for example, the patient's immune status, body weight and age. Typically, the dosage will be at a similar concentration as that found for 2′-fucosyllactose present in human breast milk. Administration may be effected continuously or intermittently such that the amount is effective for its intended purpose.

The composition may be administered individually or may be added to other compositions. For example, the composition may be added to infant formulas, nutritional compositions, rehydration solutions, maintenance or supplement compositions for the elderly or immunocompromised, or to a cocktail of various pharmaceuticals such as antibiotics, antivirals, analgesics, probiotics and anti-inflammatory agents. For example, a nutritional composition of the present invention may comprise, in addition to at least one fucose residue in an α1-2 linkage, one or more of the following components: edible macronutrients, vitamins and minerals. These components will be present in amounts which are desirable for a particular use. In particular, the amounts of such ingredients will vary depending on whether the composition is intended for use with normal, healthy infants, children or adults, or subjects having specialized need such those which accompany certain pathological conditions (e.g., metabolic disorders). The components will be of sem-purified or purified origin. By semi-purified or purified is meant a material which has been prepared by purification of a natural material or by synthesis. Such techniques are well known in the art (see, e.g., CFR for Food Ingredients and Food Processing; 10 Recommended Dietary Allowancces, 10^{th} Ed., National Academy Press, Washington, D.C., 1989).

Examples of suitable macronutrients which may be present in the nutritional composition of the present invention include edible fats, carbohydrates and proteins. Such edible fats include, for example, coconut oil, soy oil and mono- and diglycerides. Carbohydrates which may be present include, for example, glucose, edible lactose and hydrolyzed cornstarch. A protein source may be, for example, soy protein, electrodialysed whey, electrodialysed skim milk, milk whey, or hydrolysates of these proteins. All of these nutrients may be added in amounts equivalent to those present in human milk on an energy basis (i.e., on a per calorie basis).

Additionally, the nutritional compositions of the present invention may contain the following vitamins and minerals: calcium, phosphorus, potassium, sodium, chloride, magnesium, manganese, iron, copper, zinc, selenium, iodine, and Vitamins A, E, D, C, and the B complex.

The above-described compositions may be utilized in either a human or animal in order to treat or prevent various conditions or states caused by enteric microorganisms. In particular, the composition may be used against any microorganism which is thought to adhere to the host cell of relevance by way of a fucosylated receptor such as, for example, the blood group antigen H-2 or other Type II blood group antigens such as Le^{x} or Le^{y}. For example, the compositions may be used in the treatment or prevention of diarrhea, enterocolitis or necrotic enterocolitis (NEC) caused by, for example, Vibrio cholerae, enterohemorrhagic or enteropathogenic Escherichia coli, other mucosal pathogens or other enteropathogenic bacteria that bind to the H-2 receptor or to other Type II blood group antigens. The below methods may also be used in connection with all of these pathogens.

Additionally, the present invention encompasses methods for assaying or screening for the above-described compositions. In particular, the present invention encompasses four such methods. One method comprises the steps of exposing the α1,2-fucose linkage containing composition in question to the host cell receptor or to genetically-engineered cells which emulate the host cell receptor, adding the relevant microorganism of interest, and determining whether inhibition of binding of the microorganism to the host cell receptor is achieved. If so, the composition will be useful for purposes of the present invention.

The second method comprises the steps of creating a trangenic embryo whose genome will allow for expression of a composition, for example, a glycoprotein or oligosaccharide, comprising at least one fucose residue in an α1-2 linkage, waiting for birth to occur, challenging the pup with the pathogen of interest, and determining whether infection occurs. If infection does not occur, then the composition expressed by the pup is effective in that it prevents colonization by the pathogen. Such a method may also be used to screen for cross-protection. In other words, the pup may be challenged with a pathogen other than the one that the composition is thought to protect against. If infection does not result, a new indication for the composition has been discovered.

The third method comprises the steps of exposing a transfected mammalian cell line expressing neoglycoconjugates, for example, Fucα1-2Galβ1-4GlcNac, to bacteria. One then determines whether binding has occurred between the mammalian cells and the bacterial cells by use of microscopy or another biological method. Binding inhibition in solution, suspension or emulsion, as a result of the expressed composition, is then measured. More specifically, one measures the ability of the composition to prevent or reduce the binding of the bacteria to the transfected cells. The most useful compositions are those that are associated with a high percentage of binding inhibition.

With respect to the fourth method, the glycoconjugates of mammalian cells which act as pathogen receptors may be purified from naturally occurring mammalian cells or genetically engineered cells expressing glycoconjugates containing at least one fucose residue in an α1-2 linkage.

More specifically, he glycoconjugates may be immobilized on an inert support such as a resin or multi-well plates. For example, if the glycoconjugates are glycoproteins, they may be attached to derivatized agarose or activated sepharose through chemical attachment of amino radical contained in lysine or histidine residues in the protein portion. Both glycoproteins and glycolipids containing the at least one fucose residue in an α1-2 linkage may be immobilized in multi-well plates made of various materials by allowing them to dry from water solutions or solutions containing solvents such as, for example, methanol, ethanol isopropanol and chloroform. The glycoconjugates are then attached to the surfaces of the wells of the plates which can be rinsed and prepared as suitable substrates for specific bacterial attachment. The immobilized glycoconjugates containing the linkage may then be exposed to solutions containing the pathogenic bacteria which specifically binds the linkage. This bacteria may be in its native state or labeled with fluorogenic, radioactive or other labels known to those skilled in the art. Bacteria then binds to the immobilized glycoconjugates, and the binding is quantified by direct microscopic observation in the case of native bacteria, or by fluorometry, direct radioactivity counting, scintillation counting or similar techniques for labeled bacteria. Inhibitors are screened by either preincubating the native or labeled bacteria in solutions containing the inhibitors(s) or by adding inhibitor(s) in solution after bacteria and the immobilized glycoconjugates are allowed to have contact. Real inhibitors will impede attachment of bacteria to the immobilized glycoconjugates thus reducing the number of attached bacteria as determined by microscopic inspection or by any quantitative method used to ascertain and quantify the presence of bacteria.

An additional method involves creating the transgenic mammals described above. These animals are then allowed to reach maturity, and transgenic females are allowed to mate and give birth. Pups are allowed to suckle on the milk of these transgenic mammals. The pups are then challenged with the bacterial pathogen. If infection or colonization do not occur or are diminished or ameliorated in the pups, then the composition comprising at least one fucose residue in an α1-2 position, present in the milk, is protective. Thus, the composition will be useful in preventing or treating conditions caused by the pathogen.

Furthermore, the present invention includes vaccines which comprise at least one protein, for example, an adhesin, obtained from pathogenic bacteria. This protein binds to glyconjugates containing at least one fucose residue in an α1-2 linkage. This linkage may be, for example, a Fucα1-2Gal linkage. (Bacterial proteins are semi-purified or purified based upon their ability to bind to these linkages.) If desired, the adhesin may be combined with an adjuvant in order to accentuate the immune response of the host. More specifically, the vaccine may be administered parenterally in dosage-unit formulations containing standard, well-known, non-toxic physiologically acceptable adjuvants, for example, aluminum hydroxide o aluminum phosphate. The vaccine is preferably administered subcutaneously or intramuscularly. It should also be noted that a fragment of the protein or structural analog of the protein may also be utilized provided it binds to the Fucα1-2Gal linkage.

Additionally, the present invention includes a method of screening pathogenic organisms from non-pathogenic organisms. This method comprises the steps of isolating a microorganism of interest, adding a glycoconjugate receptor comprising at least on fuc α1-2 residue which binds only to pathogenic organisms, and observing whether binding occurs between the glycoconjugate and the microorganism of interest. This method can be utilized in determining whether pathogens are present in, for example, fecal, food, beverage or environmental samples.

The present invention also encompasses a method of measuring antibodies. This method comprises the steps of taking blood from a mammal, exposing the blood to a bacterial surface antigen that binds to a glyconjugate comprising at least one fuc α1-2 residue, and measuring antibody titer.

The present invention may be illustrated by the use of the following non-limiting examples:

### EXAMPLE I

### Binding of Labeled Invasive (Pathogenic) Campylobacter jejuni to Immobilized Glycoconjugates Containing Fucose Residues; Non-Binding of Invasive Strains

Bacterial binding Western blot assays were performed with digoxigenin (DIG)-labeled bacteria (Boren et al., Science 262:1892-1895 (1993)). Two ug per lane of neoglycoproteins of the blood group antigens (H-1, H-2, Le^{a,} Le^{x,} and Le-, Iso Sep AB, Sweden) were run in SDS-PAGE and transferred to nitrocellulose membranes.

Membranes were blocked with BB2 [Tris-buffered saline (TBS), pH 7.5, 1% blocking reagent (Boehringer Mannheim), 1 mM MnCl₂, 1 mM MgCl₂, and 1 mM CaCl₂] to avoid nonspecific binding. After washing 2 times in TBS, a DIG-labeled C. jejuni suspension of 0.2 OD₆₀₀ was added and incubated 4 h at room temperature with gentle stirring. Filters were Crashed 6 times for 5 min each in TBS, and incubated for 1 h with the AP-conjugated antibody to DIG, washed 5 times in TBS, and then stained. The nature of fucosylated oligosaccharides that bind Campylobacter adhesins was characterized by Western blot of immobilized neoglycoproteins. Only pathogenic strains were able to bind neoglycoproteins of the ABH (O) tissue-blood groups. A stronger binding was seen with H-2, and a greater inhibition also occurred with H-2 MAbs. Interestingly, different patterns of binding to neoglycoproteins were observed. While strains with a high cell association index bound to all the ABH (O) antigens, strains with low cell association Index bound only to H-2.

### EXAMPLE II

### Binding of Vibrio cholerae, Enteropathogenic E. coli and Enterohemorrhagic E. coli to Glycoconjugates Containing Fucosylated Residues

Vibrio cholerae 01 Ogawa strain 2995, Enterohemorrhagic Escherichia coli (EHEC) 0157:H7 strain 933, and Enteropathogenic E. coli (EPEC) 0 :H isolated from a child with diarrhea, were digoxigenin labeled. Western blots of labeled bacteria to immobilized neoglycoproteins of the blood group antigens were done using the same methods as described for Campylobacter. V. cholerae, EHEC and EPEC all readily bound to all immobilized blood group antigens. However, stronger binding was observed to H-2 and Lewis^{x}.

### EXAMPLE III

### Specific Inhibition of Binding of Vibrio cholerae to Fucosylated Neoglycoconjugates by an Anti-H-Monoclonal Antibody

To further characterize the specific binding of V. cholerae to blood group antigens, a competitive assay was developed using monoclonal antibodies (MAbs) to H-1 and H-2 blood group antigens. Immobilized H-1 and H-2 neoglycoproteins in cellulose membranes were incubated for 4 h at room temperature with a suspension of DIG-labeled V. cholerae (at a bacterial concentration of 0.01 OD₆₀₀ ) and anti-H-1 and -H-2 MAbs at 1:10 and 1:20 dilutions. As a control, the first lane of immobilized blood group antigens was incubated with the DIG-labeled bacteria and phosphate buffered saline.

Membranes were Lashed 6 times with TRIS buffered saline, pH 7.1, and anti-DIG-alkaline phosphatase conjugate was added and incubated for 1 h, washed 5 times, and a substrate, phosphate X-NBT-TBS, pH 9.5, was added. Membranes were washed and dried. Binding of DIG-labeled V. cholerae was inhibited only with anti-H-2 S even at a low dilution, but no inhibition of binding occurred with anti-H-2 MAbs.

### EXAMPLE IV

### Binding of Vibrio cholerae to CHO Cells Transfected With the Human α1-2 Fucosyltransferase H Expressing Fuc α1-2 Glycoconjugates and Lack of Binding to Native CHO Cells

To assess the specific binding of V. cholerae to blood group antigens, a modified method previously described for bacterial cell association was used (Cravioto et al., J. Infect. Dis. 163:1247-1255 (1991)). Transfected CHO cells expressing α 1-2 residues were grown to confluency in 10% fetal calf serum in modified Eagle's medium. Concomitantly, as a control, parental, non-transfected CHO cells which do not express α 1-2 fucosyl residues, were also cultured in the same manner.

Confluent monolayers were detached with 2 mL of 0.025% trypsin-EDTA, and viable cells counted using 0.05% trypan blue; 0.4 mL of a suspension of 2×10⁵ viable cells/mL were seeded into each well of an 8-chamber slide (Lab-Tek, Miles Scientific; Naperville, Ill.) and incubated for 18 hr. Cells were washed in Hank's PBS and 100 uL of the V. cholerae suspension containing approximately 9×10⁸ bacteria per mL in MEM with 1% fetal calf serum were added to each chamber and incubated at 37° C. under 8% CO₂ for 1, 2, 3, 4, 5, and 6 hr. Wells were rinsed 6 times with PBS, fixed with 10% formalin for 1 hr, stained by the Warthin-Starry method, and examined under oil immersion with light microscopy. Results were expressed as percent of cells with associated Campylobacter organisms. One hundred cells per well were counted. A cell was considered to be Campylobacter-associated if three or more bacteria were seen attached to it. For standardization, each reference strain was tested 5 times on different days.

For scanning electron microscopy, V. cholerae infected CHO cells were preserved in cacodylate for further processing.

When V. cholerae was incubated with parental, non-transfected CHO cells no binding occurred, while, when incubated with transfected CHO cells expressing α 1-2 residues, abundant bacteria clumped and covered the whole cell surface. Scanning electron microscopy showed a uniform attachment of bacteria Lo the cell surface without any evident initial damage, and clumping of bacteria also was seen.

### EXAMPLE V

### Binding of Vibrio cholerae to Transfected CHO Cells with Dose-Dependent Kinetics

To define the ability of V. cholerae to attach to transfected CHO cells and to determine the effect of bacterial concentration with time, experiments were done using inocula of 10⁴ and 10⁶ CFU/mL. Bacterial attachment to cells was assessed every 30 min up to 1.5 h. After this time, cell lysis occurred rapidly and cell attachment could not be further evaluated. Bacterial cell attachment started as early as 30 min and reached a peak a 90 min. Bacterial cell attachment was dose-dependent and proportional to the inoculum; the greater the inoculum, the higher the number of bacteria attached.

### EXAMPLE VI

### Inhibition of Binding of V. cholerae and E. coli to Transfected Cell Lines by 2′Fucosyllactose in a Dose-Dependent Manner

To determine whether 2′Fucosyllactose is an important chemical structure of the cell receptor for V. cholerae and enterohemorrhagic E. coli (EHEC), a competitive assay was set up. A volume of 400 ul of a suspension of transfected CHO (α1-2 FT) cells at a concentration of 4×10⁵ cells/mL was placed in each well of an 8-well microchamber slide (Lab-Tek, Nunc, Inc. Naperville, Ill.). Cells were incubated overnight at 37° C. in a 5% CO₂ atmosphere. A bacterial suspension of 1.5×10⁸ of V. cholerae was added with either 0.5, 1, or 2 mg/mL of 2′fucosyllactose and incubated at 37° C. Wells were washed 6 times with PBS. Cells were detached with 25 ml of triton x 100 and serial dilutions in TBS or McConkey agar plates were done for colony counting. A significant inhibition of bacterial cell attachment was observed with a concentration as low as 0.5 mg/ml of 2′fucosyllactose for both V. cholerae and EHEC. A dose-dependent inhibition occurred; however, a greater inhibition of binding occurred with EHEC.

### EXAMPLE VII

### A Specific Adhesin of Vibrio cholera Identified in a Western Blot by Binding to Labeled H-2 Antigen Neoglycoconjugate

Extraction of outer membrane proteins (OMPs) of V. cholerae: OMPs were extracted by the Sarkosyl method as previously described with some modifications (McCoy et al., Infect. Immun. 11:517-525 (1975)). Briefly, 1 g (wet weight) of washed cells was resuspended in HEPES 10 mM buffer (pH 7.4), washed twice and sonicated 4 times. MgCl (1M) was added with Dnase and Rnase (1 mg/ml)and sonicated twice. Intact cells were removed by centrifugation at 5,000×g for 30 min. The supernatant was centrifuged for 45 min at 120,000×g. The pellet was then suspended in 1 ml of sterile HEPES. Pellets were pooled and centrifuged at 120,000×g for 45 min. The pellet was resuspended in HEPES-Sarkosyl at 20% in a ratio of 1:6 of membrane protein:Sarkosyl, shaken for 30 min at room temperature, and centrifuged at 150,000×g for 45 min. The pellet was suspended in 5 mil EDTA-Tris buffer (pH 7.8), incubated for 20 min at 37° C., and centrifuged at 150,000×g for 45 min. The resulting pellet was suspended in 1 ml of sterile HEPES and stored at −70° C.

Conditions of SDS-PAGE run: Protein samples were run in 7.5% polyacrylamide gels under reducing conditions by the standard procedure of Laemmli. Gels w,ere stained with Coomassie blue stain to visulalize the protein bands.

DIG-labeling of H-2 neoglycoconjugate: One mg of H-2 neoglycoprotein was dissolved in 1 ml of PBS, pH 8.5, supplemented with aprotinin (2 ug/ml). 8.18 ul of a 40 mg/ml solution of Digoxigenin-3-0-mehylcarbonyl-e-aminocaproic acid-N-hydroxy-succinimide ester in DMSO was added to the initial solution and the resulting mixture was incubated for 2 hr at room temperature. The protein solution was dialyzed against PBS.

Conditions for the Western blot: Proteins were run in SDS-PAGE with 5 ug per lane and transferred to nitrocellulose. Membranes were blocked with BB2 [Tris-buffered saline (TBS), pH 7.5, 1% blocking reagent (Boehringer-Mannheim), 1 mM MnCl₂, 1 MM MgCl₂, and 1 mM CaCl₂] to avoid nonspecific binding. After being washed 2 times in TBS, individual strips were cut from the membrane and reacted With appropriate concentration of H2-neoglycoprotein labeled with DIG (1, 2, 4 and 8 ug) for 4 h at room temperature with gentle stirring. Strips were washed 6 times for 5 min each in TBS, incubated for 1 h with the AP-conjugated antibody to DIG, washed 5 times in TBS, and then developed with x-phosphate/NBT in TBS, pH 9.5.

DIG-labeled H-2 neoglycoconjugate readily bound at increasing concentration to a single protein band of approximately 33 kDa. Data suggest that there is a surface protein of V. cholerae that acts as a lectin-like protein with specific affinity to H-2 tissue/blood group antigen.

1. A pharmaceutical composition comprising at least one fucose residue in an α1-2 linkage and a pharmaceutically acceptable carrier.

2. The pharmaceutical composition of claim 1 wherein said at least ore fucose residue in an α1-2 linkage is present in a compound selected from the group consisting of 2′-fucosyllactose, difucosyllactose, Fucα1-2Galβ1-4[Fucα1-3]Glc, glycoproteins or glycodeptides containing the structure Fucα1-2Galβ1-4Glc Nac 31-3 GM-Fuc (Fucα1-2Galβ1-3GalNac), glycolipids and fucosylated derivatives of neutral glycolipids.

3. A nutritional composition comprising at least one fucose residue In an α1-2 linkage, at least one protein not found in human breast milk, and at least one member selected from the group consisting of an edible fat, a carbohydrate, a protein, a vitamin and a mineral.

4. The nutritional composition of claim 3 wherein said at least one fucose residue in an α1-2 linkage is present in a compound selected from the group consisting of 2′-fucosyllactose, difucosyllactose, Fucα1-2Galβ1-4[Fucα1-3]Glc, glycoproteins or glycopeptides containing the structure Fucα1-2Galβ1-4Glc Nac β1-3 GMj-Fuc (Fucα1-2Galβ1-3GalNac), glycolipids and fucosylated derivatives of neutral glycolipids.

5. The nutritional composition of claim 4 wherein said composition is an infant formula.

6. A rehydration solution comprising said composition of claim 1 or claim 3.

7. A method of preventing or treating diarrhea or enterocolitis in a patient comprising administering a composition comprising at least one fucose residue in an α1-2 linkage to a patient in need of said prevention or treatment, said composition being administered in an amount sufficient to effect said treatment of prevention.

8. The method of claim 7 wherein said composition is administered to a human or to an animal.

9. The method of claim w Wherein said diarrhea or enterocolitis is caused by a microorganism selected from the group consisting of Escherichia coli and Vibrio cholerae.

10. The method of claim 9 wherein said enterocolitis is necrotic enterocolitis.

11. A method of screening for a composition which prevents the attachment of E. coli or V. cholerae to a host cell receptor comprising the steps of:
a) exposing said composition in question to said host cell receptor;
b) adding Escherichia coli or Vibrio cholerae to said composition of step (a) and said host cell receptor; and
c) determining whether Inhibition of binding of said cells to said host cell receptor occurs, said inhibition indicating the presence of a composition which binds to said host cell receptor and prevents attachment of said E. coli or V. cholerae to said host cell receptor.

12. The method of claim 1 wherein said host cell receptor comprises a fucosylated blood group antigen.

13. The method of claim 12 wherein said fucosylated blood group antigen is H-2.

14. A vaccine comprising at least one protein which binds to at least one fucose residue in an α1-2 linkage and a physiologically acceptable adjuvant.

15. The vaccine of claim 14 wherein said vaccine is administered subcutaneously or Intramuscularly.

16. A method of screening for a composition which prevents the attachment of E. coli or V. cholerae to a mammalian cell receptor comprising the steps of:
a) constructing a transgenic mammalian embryo which, upon birth, produces a composition comprising at least one fucose residue in an α1-2 linkage;
b) implanting said trangenic mammalian embryo into a recipient adult female;
c) allowing gestation and birth to occur;
d) challenging said resulting mammal with E. coli or V. cholerae; and
e) determining whether infection develops in said resulting mammal, lack of infection indicating that said composition expressed by said resulting mammal prevents attachment of E. coli or V. cholerae to said receptor of said resulting mammal.

17. A method of screening for a composition which prevents the attachment of E. coli or V. cholerae to a host cell receptor comprising the steps of:
a) exposing transfected, mammalian cells expressing a neoglyconjugate to E. coli or V. cholerae;
b) determining whether binding has occurred between said mammalian cells and said E. coli or V. cholerae, a high degree of binding inhibition relative to a control indicating that said neoglyconjugate presents attachment of said E. coli or V. cholerae to said receptor of said mammalian cells.

18. A method of screening for a composition which prevents the attachment of E. coli or V. cholerae to a host cell receptor comprising she seeps of:
a) purifying a glycoconjugate comprising at least one fucose residue in an α1-2 linkage from a mammalian cell;
b) immobilizing said glycoconjugate on a solid support;
c) exposing said immobilized glycoconjugate to E. coli cells or V. cholerae cells;
d) adding a composition of interest to said immobilized glycoconjugate and E. coli cells or V. cholerae cells;
d) determining whether binding occurs between said immobilized glycoconjugate and said E. coli cells or V. cholerae cells, lack of binding indicating a composition which prevents the attachment of E. coli cells or V. cholerae cells to a host cell receptor.

19. A method c screening for a composition which prevents the attachment of E. coli or V. cholerae to a receptor of mammalian cells comprising the steps of:
a) constructing a transgenic, mammalian embryo which, after birth, produces a composition comprising at least one fucose residue in an α1-2 linkage;
b) implanting said transgenic, mammalian embryo into a recipient female;
c) allowing gestation and birth to occur;
d) allowing said resulting transgenic mammal to mate and produce offspring;
e) allowing said offspring to suckle on milk produced by said transgenic mammals;
f) challenging said offspring with E. coli cells or V. cholerae cells;
g) determining whether infection occurs, lack of infection indicating a composition present in said milk of said transgenic mammal which prevents the attachment of E. coli or V. cholerae to a receptor of cells of said offspring.

20. A method of screening pathogenic microorganisms from non-pathogenic microorganisms comprising the steps of:
a) isolating a microorganism of interest;
b) exposing said microorganism to a glyconjugate receptor comprising at least one fucose residue in an α1-2 linkage, wherein said receptor binds only to pathogenic microorganisms; and
c) determining whether binding occurs between said glycoconjugate receptor and said microorganism of interest, binding indicating that said microrganism is pathogenic and non-binding indicating that said microorganism is non-pathogenic.
